(19) United States
(12) Patent Application Publication
(10) Pub. No.: US 2013/0090320 A1
(43) Pub. Date: Apr. 11, 2013

(54) METHOD OF TREATING PROSTATE CANCER

(71) Applicant: NIIKI PHARMA INC., Hoboken, NJ (US)

(72) Inventor: Hooshmand SHESHBARADARAN, Hoboken, NJ (US)

(73) Assignee: NIIKI PHARMA INC., Hoboken, NJ (US)

(21) Appl. No.: 13/657,888

(22) Filed: Oct. 23, 2012

Related U.S. Application Data
(63) Continuation of application No. PCT/US2011/033509, filed on Apr. 22, 2011.
(60) Provisional application No. 61/327,492, filed on Apr. 23, 2010.

(51) Int. Cl. A61K 31/555

(52) U.S. Cl. CPC A61K 31/555
USPC 514/186

(57) ABSTRACT

A therapeutic method for treating prostate cancer is disclosed comprising administering to a patient in need of treatment a ruthenium complex salt.

### CROSS-REFERENCE TO RELATED APPLICATIONS

This application is a continuation of PCT/US11/33509 filed on Apr. 22, 2011, which claims the benefit of U.S. Provisional Application No. 61/327,492 filed on Apr. 23, 2010.

### FIELD OF THE INVENTION

The present invention generally relates to methods for treating cancer, and particularly to a method of treating prostate cancer.

### BACKGROUND OF THE INVENTION

Prostate cancer is the most common type of cancer in men in the United State. For example, in 2008, there were 186,000 new cases of prostate cancer, and 28,600 deaths caused by prostate cancer. Thus, there is a significant need for new agents in treating brain cancer.

Both Peti et al, Eur. J. Inorg. Chem. 1999, 1551-1555 and PCT Publication No. WO2008/154553 disclose methods of synthesizing the compound sodium trans-[tetrachlorobis(1H-indazole)ruthenate(III)].

### SUMMARY OF THE INVENTION

It has now been discovered that the compound sodium trans-[tetrachlorobis(1H-indazole)ruthenate(III)] is especially effective in inhibiting prostate cancer cell growth. It has also been surprisingly discovered that the compound sodium trans-[tetrachlorobis(1H-indazole)ruthenate(III)] is equally effective in prostate cancer cells both sensitive and resistant to drugs such as docetaxel and vinblastine.

Accordingly, in a first aspect, the present invention provides a method of treating prostate cancer, which comprises treating a patient identified as having prostate cancer, with a therapeutically effective amount of trans-[tetrachlorobis(1H-indazole)ruthenate(III)] or a pharmaceutically acceptable salt thereof.

In a second aspect, the present invention provides a method of preventing or delaying the onset of prostate cancer, comprising administering to a patient identified to be in need of prevention, or delaying the onset, of prostate cancer a prophylatically effective amount of trans-[tetrachlorobis(1H-indazole)ruthenate(III)] or a pharmaceutically acceptable salt thereof.

The present invention further provides use of trans-[tetrachlorobis(1H-indazole)ruthenate(III)] or a pharmaceutically acceptable salt thereof for the manufacture of a medicament useful for treating, preventing or delaying the onset of prostate cancer.

In yet another aspect, the present invention provides a method of treating refractory prostate cancer comprising identifying a patient having refractory prostate cancer and treating the patient with a therapeutically effective amount of trans-[tetrachlorobis(1H-indazole)ruthenate(III)] or a pharmaceutically acceptable salt thereof. In specific embodiments, the patient has a prostate cancer that is refractory to a treatment comprising docetaxel and/or vinblastine.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a graph showing the dose-dependent growth inhibition by sodium trans-[tetrachlorobis(1H-indazole)ruthenate(III)] (MTT assay) in LNCaP cells; and

FIG. 2 is a graph showing the dose-dependent growth inhibition by sodium trans-[tetrachlorobis(1H-indazole)ruthenate(III)] (MTT assay) in PC-3 cells.

### DETAILED DESCRIPTION OF THE INVENTION

The present invention is at least in part based on the discovery that the compound sodium trans-[tetrachlorobis(1H-indazole)ruthenate(III)] is effective in treating prostate cancer. Accordingly, in accordance with a first aspect of the present invention, a method is provided for treating prostate cancer. Specifically, the method comprises treating a patient having prostate cancer with a therapeutically effective amount of trans-[tetrachlorobis(1H-indazole)ruthenate(III)] or a pharmaceutically acceptable salt thereof. That is, the present invention is directed to the use of trans-[tetrachlorobis(1H-indazole)ruthenate(III)] or a pharmaceutically acceptable salt thereof for the manufacture of medicaments for treating prostate cancer in patients identified or diagnosed as having prostate cancer. In preferred embodiments, an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]) or indazolium trans-[tetrachlorobis(1H-indazole)ruthenate(III)] is used.

In the various embodiments of this aspect of the present invention, the treatment method optionally also comprises a step of diagnosing or identifying a patient as having prostate cancer. The identified patient is then treated with or administered with a therapeutically effective amount of trans-[tetrachlorobis(1H-indazole)ruthenate(III)] or a pharmaceutically acceptable salt thereof (e.g., sodium trans-[tetrachlorobis(1H-indazole)ruthenate(III)] or indazolium trans-[tetrachlorobis(1H-indazole)ruthenate(III)]). Various prostate cancers can be diagnosed in any conventional diagnostic methods known in the art including PSA test, biopsy, Gleason score, PCA-3 mRNA test, etc.

In addition, it has also been surprisingly discovered that the compound sodium trans-[tetrachlorobis(1H-indazole)ruthenate(III)] is equally effective in prostate cancer cells resistant to tubulin inhibitors such as docetaxel and vinblastine. Accordingly, the present invention also provides a method of treating refractory prostate cancer comprising treating a patient identified as having refractory prostate cancer with a therapeutically effective amount of trans-[tetrachlorobis(1H-indazole)ruthenate(III)] or pharmaceutically acceptable salt thereof (e.g., sodium trans-[tetrachlorobis(1H-indazole)ruthenate(III)] or indazolium trans-[tetrachlorobis(1H-indazole)ruthenate(III)]). In one embodiment, the patient has a prostate cancer that is refractory to a treatment comprising a taxane (e.g., docetaxel, paclitaxel, abraxane, etc.). In another embodiment, the patient has a prostate cancer that is refractory to a treatment comprising a vinca alkaloid (e.g., vinblastine, vincristine, vindesine and vinorelbine). That is, the present invention is also directed to the use of trans-[tetrachlorobis(1H-indazole)ruthenate(III)] or a pharmaceutically acceptable salt thereof (e.g., sodium trans-[tetrachlorobis(1H-indazole)ruthenate(III)] or indazolium trans-[tetrachlorobis(1H-indazole)ruthenate(III)]) for the manufacture of medicaments for treating refractory prostate cancer, e.g., a prostate cancer refractory to a taxane (e.g., docetaxel, paclitaxel, abraxane, etc.) or a vinca alkaloid (e.g., vinblastine, vincristine, vindesine and vinorelbine).

The term “refractory prostate cancer,” as used herein refers to prostate cancer that either fails to respond favorably to an anti-neoplastic treatment that does not include trans-[tetrachlorobis(1H-indazole)ruthenate(III)] or a pharmaceutically acceptable salt thereof, or alternatively, recurs or relapses after responding favorably to an antineoplastic treatment that does not include trans-[tetrachlorobis(1H-indazole)ruthenate(III)] or a pharmaceutically acceptable salt thereof. Accordingly, “a prostate cancer refractory to a treatment” as used herein means a prostate cancer that fails to respond favorably to, or resistant to, the treatment, or alternatively, recurs or relapses after responding favorably to the treatment. For example, such a prior treatment may be a chemotherapy regimen including docetaxel and estramustine phosphate.

Thus, in some embodiments, in the method of the present invention, trans-[tetrachlorobis(1H-indazole)ruthenate(III)] or a pharmaceutically acceptable salt thereof is used to treat prostate cancer patients having a tumor that exhibits resistance to a treatment comprising one or more drugs such as a taxane (e.g., docetaxel, paclitaxel, abraxane, etc.) and vinca alkaloids (e.g., vinblastine, vincristine, vindesine and vinorelbine). In other words, the method is used to treat a prostate cancer patient having previously been treated with a treatment regimen that includes one or more drugs such as taxane (e.g., docetaxel, paclitaxel, abraxane, etc.) and vinca alkaloids (e.g., vinblastine, vincristine, vindesine and vinorelbine), and whose prostate cancer was found to be non-responsive to the treatment regimen or have developed resistance to the treatment regimen. In other embodiments, the method is used to treat a prostate cancer patient previously treated with a treatment comprising one or more drugs such as taxanes (e.g., docetaxel, paclitaxel, abraxane, etc.) and vinca alkaloids (e.g., vinblastine, vincristine, vindesine and vinorelbine), but the prostate cancer has recurred or relapsed, that is, a prostate cancer patient who has previously been treated with one or more such drugs, and whose cancer was initially responsive to the previously administered one or more such drugs, but was subsequently found to have relapsed. In specific embodiments, sodium trans-[tetrachlorobis(1H-indazole)ruthenate(III)] is used to treat prostate cancer patients previously treated with docetaxel, i.e., who have a tumor that exhibits resistance to, or relapsed after a treatment including docetaxel. In other specific embodiments, sodium trans-[tetrachlorobis(1H-indazole)ruthenate(III)] is used to treat prostate cancer patients previously treated with a vinca alkaloid, i.e., who have a prostate cancer that exhibits resistance to, or relapsed after a treatment including a vinca alkaloid.

To detect a refractory prostate cancer, patients undergoing initial treatment can be carefully monitored for signs of resistance, non-responsiveness or recurring prostate cancer. This can be accomplished by monitoring the patient's cancer's response to the initial treatment which, e.g., may include docetaxel or vinblastine. The response, lack of response, or relapse of the cancer to the initial treatment can be determined by any suitable method practiced in the art. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” criteria as described in detail in Therasse et al, J. Natl. Cancer Inst. 92:205-216 (2000).

In accordance with yet another aspect of the present invention, a method is provided for preventing or delaying the onset of prostate cancer, or preventing or delaying the recurrence of prostate cancer, which comprises treating a patient in need of the prevention or delay with a prophylatically effective amount of trans-[tetrachlorobis(1H-indazole)ruthenate(III)] or a pharmaceutically acceptable salt thereof (e.g., sodium trans-[tetrachlorobis(1H-indazole)ruthenate(III)] or indozolium trans-[tetrachlorobis(1H-indazole)ruthenate(III)]).

For purposes of preventing or delaying the recurrence of prostate cancer, prostate cancer patients who have been treated and are in remission or in a stable or progression free state may be treated with a prophylatically effective amount of trans-[tetrachlorobis(1H-indazole)ruthenate(III)] or a pharmaceutically acceptable salt thereof (e.g., sodium trans-[tetrachlorobis(1H-indazole)ruthenate(III)] or indazolium trans-[tetrachlorobis(1H-indazole)ruthenate(III)]) to effectively prevent or delay the recurrence or relapse of prostate cancer.

As used herein, the phrase “treating . . . with . . . ” or a paraphrase thereof means administering a compound to the patient or causing the formation of a compound inside the body of the patient.

In accordance with the method of the present invention, prostate cancer can be treated with a therapeutically effective amount of trans-[tetrachlorobis(1H-indazole)ruthenate(III)] or a pharmaceutically acceptable salt thereof (e.g., sodium trans-[tetrachlorobis(1H-indazole)ruthenate(III)] or indazolium trans-[tetrachlorobis(1H-indazole)ruthenate(III)]) alone as a single agent, or alternatively in combination with one or more other anti-cancer agents. Example of pharmaceutically acceptable salts include alkali metal salts (e.g., sodium or potassium salt), indazolium salts, etc. An alkali metal salt, preferably sodium salt of trans-[tetrachlorobis(1H-indazole)ruthenate(III)] (i.e., sodium trans-[tetrachlorobis(1H-indazole)ruthenate(III)] or potassium trans-[tetrachlorobis(1H-indazole)ruthenate(III)]) is particularly useful.

Alkali metal salts of trans-[tetrachlorobis(1H-indazole)ruthenate(III)] can be made in any methods known in the art. For example, PCT Publication No. WO/2008/154553 discloses an efficient method of making sodium trans-[tetrachlorobis(1H-indazole)ruthenate(III)]. U.S. Pat. No. 7,338,946 discloses indazolium trans-[tetrachlorobis(1H-indazole)ruthenate(III)]) and a formulation containing the indazolium salt.

The pharmaceutical compounds such as a pharmaceutically acceptable salt of trans-[tetrachlorobis(1H-indazole)ruthenate(III)] (e.g., sodium trans-[tetrachlorobis(1H-indazole)ruthenate(III)], or indazolium trans-[tetrachlorobis(1H-indazole)ruthenate(III)]) can be administered through intravenous injection or any other suitable means at an amount of from 0.1 mg to 1000 mg per kg of body weight of the patient based on total body weight. The active ingredients may be administered at once, or may be divided into a number of smaller doses to be administered at predetermined intervals of time, e.g., once daily or once every two days. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

The present invention also provides a use of trans-[tetrachlorobis(1H-indazole)ruthenate(III)] or a pharmaceutically acceptable salt thereof (e.g., an alkali metal salt of trans-[tetrachlorobis(1H-indazole)ruthenate(III)] such as sodium trans-[tetrachlorobis(1H-indazole)ruthenate(III)] or potassium trans-[tetrachlorobis(1H-indazole)ruthenate(III)], or indazolium trans-[tetrachlorobis(1H-indazole)ruthenate(III)]) for the manufacture of a medicament useful for treating prostate cancer.

In specific embodiments, the present invention provides a use of trans-[tetrachlorobis(1H-indazole)ruthenate(III)] or a pharmaceutically acceptable salt thereof (e.g., an alkali metal salt of trans-[tetrachlorobis(1H-indazole)ruthenate(III)] such as sodium trans-[tetrachlorobis(1H-indazole)ruthenate(III)] or potassium trans-[tetrachlorobis(1H-indazole)ruthenate(III)], or indazolium trans-[tetrachlorobis(1H-indazole)ruthenate(III)]) for the manufacture of a medicament useful for treating prostate cancer previously treated with docetaxel and/or vinblastine, i.e., prostate cancer that exhibits resistance to, or relapsed after a treatment including docetaxel and/or vinblastine.

The medicament can be, e.g., in an injectable form, e.g., suitable for intravenous, intradermal, or intramuscular administration. Injectable forms are generally known in the art, e.g., in buffered solution or suspension.

In accordance with another aspect of the present invention, a pharmaceutical kit is provided comprising in a container a unit dosage form of a compound containing trans-[tetrachlorobis(1H-indazole)ruthenate(III)], or a pharmaceutically acceptable salt thereof (e.g., an alkali metal salt of trans-[tetrachlorobis(1H-indazole)ruthenate(III)] such as sodium trans-[tetrachlorobis(1H-indazole)ruthenate(III)] or potassium trans-[tetrachlorobis(1H-indazole)ruthenate(III)], or indazolium trans-[tetrachlorobis(1H-indazole)ruthenate(III)]), and optionally instructions for using the kit in the methods in accordance with the present invention, e.g., treating, preventing or delaying the onset of prostate cancer, or preventing or delaying the recurrence of prostate cancer, or treating refractory prostate cancer. As will be apparent to a skilled artisan, the amount of a therapeutic compound in the unit dosage form is determined by the dosage to be used on a patient in the methods of the present invention. In the kit, a compound having trans-[tetrachlorobis(1H-indazole)ruthenate(III)] or a pharmaceutically acceptable salt thereof (e.g., an alkali metal salt of trans-[tetrachlorobis(1H-indazole)ruthenate(III)] such as sodium trans-[tetrachlorobis(1H-indazole)ruthenate(III)] or potassium trans-[tetrachlorobis(1H-indazole)ruthenate(III)], or indazolium trans-[tetrachlorobis(1H-indazole)ruthenate(III)]) can be in lyophilized form in an amount of, e.g., 25 mg, in an ampoule. In the clinic, the lyophilized form can be dissolved in a buffer and administered to a patient in need of the treatment in accordance with the present invention.

### Example 1

To test the activities of sodium trans-[tetrachlorobis(1H-indazole)ruthenate(III)], ATCC's MTT Cell Proliferation Assay® was performed using human prostate cancer cell lines LNCaP clone FGC (prostate carcinoma) and PC-3 (Prostate carcinoma). Stock cultures were allowed to grow to 70-80% confluence for this study. The anti-proliferative activity of sodium trans-[tetrachlorobis(1H-indazole)ruthenate(II)], against the indicated cell lines was evaluated in vitro using the ATCC's MTT Cell Proliferation Assay (Catalog No. 30-1010K). LNCaP clone FGC plates were seeded with 4,000 cells/well, and the cells were grown in RPMI 1640 medium containing 1% HEPES (1M solution), 1% sodium pyruvate, 1% glucose (45% solution), 10% heat-inactivated FBS and 1% penicillin/strep/glutamine. PC-3 plates were seeded with 6,000 cells/well, and the cells were grown in Ham's F12 medium containing 10% heat-inactivated FBS, and 1% penicillin/strep/glutamine. Cultures were maintained in a 37° C. humidified 5% CO₂/95% air atmosphere. The cells were treated with sodium trans-[tetrachlorobis(1H-indazole)ruthenate(III)] at 1,000 μM, or a series of 4× dilutions thereof (250 μM, 62.5 μM, etc.). 100 μl of medium was removed from each well at 72 hours post-treatment and 10 μl MTT reagent was added to each well. The plates were incubated at 37° C. for 4 hours and then 100 μl of detergent was added. The plates were left overnight at room temperature in the dark and was read on a plate reader using SoftMax® Pro (version 5.2, Molecular Devices).

The absorbance data was analyzed as follows: Absorbance values were converted to Percent of Control and plotted against test agent concentrations for IC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration versus Percent of Control were analyzed using the 4-parameter equation to obtain IC₅₀ values and other parameters that describe the sigmoidal dose response curve.

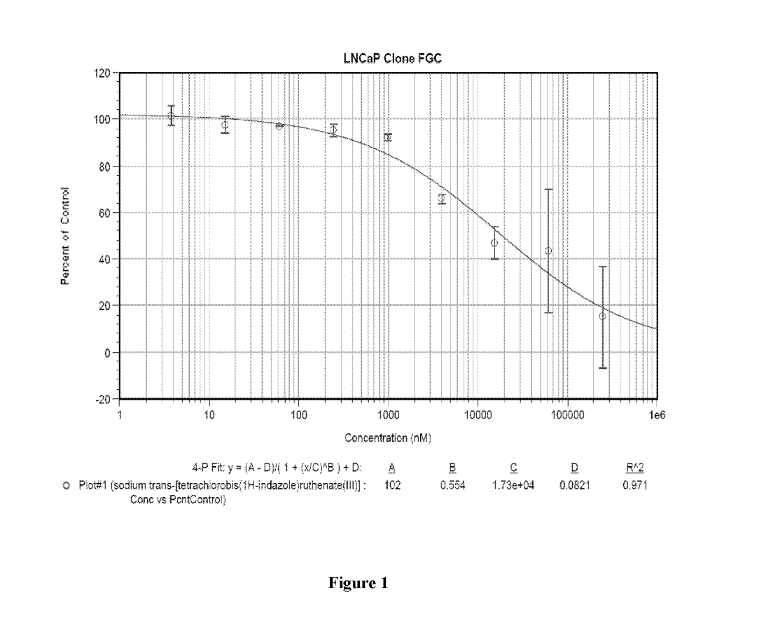

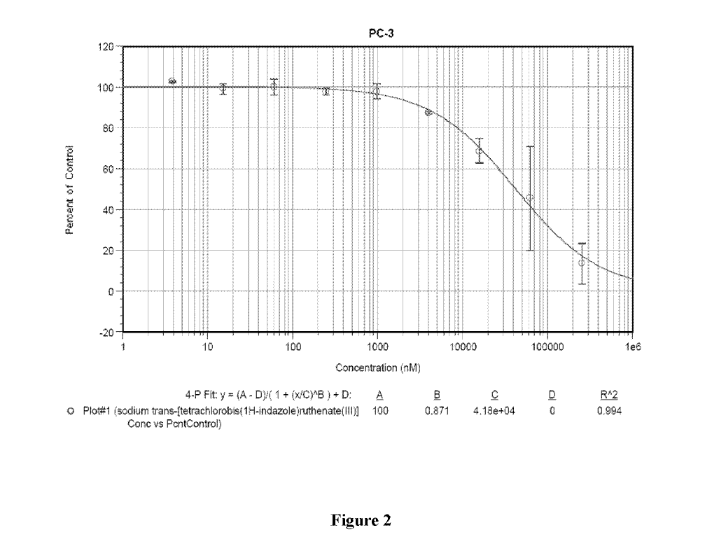

The IC₅₀ value for the test agent was estimated by curve-fitting the data using the following four parameter-logistic equation:

$$Y = \frac{Top - Bottom}{1 + {( \frac{X}{{IC}_{50}} )}^{n}} + Bottom$$

wherein “Top” is the maximal % of control absorbance (100%) (Value “A” in FIGS. 1 and 2), “Bottom” is the minimal % of control absorbance at the highest agent concentration (down to zero) (Value “D” in FIGS. 1 and 2), Y is the Percent of Control absorbance, X is the test agent Concentration, IC₅₀ is the concentration of agent that inhibits cell growth by 50% compared to the control cells (Value “C” in FIGS. 1 and 2), n is the slope of the curve (Value “B” in FIGS. 1 and 2). The IC₅₀ of sodium trans-[tetrachlorobis(1H-indazole)ruthenate(III)] was 17.3 μM in LNCaP cell line (FIG. 1), and 41.8 μM in PC-3 cell line (FIG. 2).

In addition, the IC₅₀ values of docetaxel in the LNCaP and PC-3 cell lines were obtained from published literature. The ratios of IC₅₀ values of sodium trans-[tetrachlorobis(1H-indazole)ruthenate(III)] in a cell line sensitive to one of the other drugs and a cell line insensitive to the same drug were calculated. The results are shown in Table 1 below (“Test Drug” in the table denotes sodium trans-[tetrachlorobis(1H-indazole)ruthenate(III)]). The data shows that sodium trans-[tetrachlorobis(1H-indazole)ruthenate(III)] is in fact more effective in LNCaP cells that are resistant to docetaxel and vinblastine than in PC-3 cells that are relatively sensitive to docetaxel and vinblastine. See Domingo-Domenech et al., Clin. Cancer Res., 12(18):5578-5586 (2006); Blagosklonny et al., J. Urol. 163(3):1022-6 (2000). As such, sodium trans-[tetrachlorobis(1H-indazole)ruthenate(III)] is potentially effective in treating prostate cancer resistant to such other drugs.

TABLE 1

| | Test Drug | | Docetaxel* | | |
|---|---|---|---|---|---|
| | | IC₅₀ Ratio | | | IC₅₀ Ratio |
| Cell Line | IC₅₀ | (LNCaP/PC-3) | IC₅₀ | | (LNCaP/PC-3) |
| PC-3 | 41.8 μM | 0. 41 | 2.5-5 | nM | 12-20 |
| LNCaP | 17.3 μM | | 50-60 | nM | |

*Domingo-Domenech et al., Clin. Cancer Res., 12(18):5578-5586 (2006).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

What is claimed is:

1. A method for treating prostate cancer, comprising administering to a patient in need of treatment a therapeutically effective amount of a pharmaceutically acceptable salt of trans-[tetrachlorobis(1H-indazole)ruthenate(III)].

2. The method of claim 1, wherein the prostate cancer is a refractory prostate cancer.

3. The method of claim 1, wherein said pharmaceutically acceptable salt is an alkali metal salt of trans-[tetrachlorobis(1H-indazole)ruthenate(III)].

4. The method according to claim 1, wherein said pharmaceutically acceptable salt is sodium trans-[tetrachlorobis(1H-indazole)ruthenate(III)].

5. The method of claim 2, wherein said prostate cancer is refractory to a treatment comprising a taxane, or a vinca alkaloid.

6. The method of claim 5, wherein said prostate cancer was previously treated with a regimen comprising docetaxel.

7. The method of claim 5, wherein said prostate cancer was previously treated with a regimen comprising vinblastine.
